(19) United States
(12) Patent Application Publication
(10) Pub. No.: US 2026/0257023 A1
(43) Pub. Date: Sep. 3, 2026

(54) DIAMETER EXTENSION DEVICE FOR MEDICAMENT DELIVERY DEVICE

(71) Applicant: SHL Medical AG, Zug (CH)

(72) Inventors: Wen-Chung Tsai, Nacka Strand (SE); Orshano Kourkis, Nacka Strand (SE); René Zander, Zug (CH); Hsuan Wang, Zug (CH); Lukas Bannwart, Zug (CH); Núria De Santiago Giner, Zug (CH)

(21) Appl. No.: 19/163,923

(22) PCT Filed: Mar. 4, 2024
(86) PCT No.: PCT/EP2024/055605

§ 371 Date: Sep. 10, 2025

(30) Foreign Application Priority Data
Mar. 14, 2023 (EP) 23161897.6

(51) Int. Cl. A61M 5/32; A61M 5/20

(52) U.S. Cl. CPC A61M 5/326; A61M 5/20; A61M 2005/2013; A61M 2005/3267

(57) ABSTRACT

An embodiment of the present disclosure relates to a diameter extension device for a medicament delivery device. The medicament delivery device includes a housing body with a generally tubular structure with a central axis in a proximal-to-distal direction and configured to hold a medicament delivery system and a drive assembly. The medicament delivery device further includes a delivery member cover configured to cover at least a proximal portion of the medicament delivery system. The delivery member cover is further configured to move, when pressed against tissue at an injection site, coaxially with the central axis within the housing body from a first proximal position to at least a second more distal position, and is further configured to activate by movement from the first position towards the second position the drive assembly to initiate medicament delivery. The medicament delivery device further includes at least two structural features recessing or protruding from an outer surface of the housing body. The diameter extension device comprises: a body with a generally tubular structure being configured to surround at least a portion of the housing body and to move axially in direction of the central axis on the outer surface of the housing body, and at least two guiding means configured to communicate with the at least two structural features of the housing body in order to define the axial movement of the diameter extension device on the outer surface of the housing body.

### TECHNICAL FIELD

The present invention relates to a diameter extension device for a medicament delivery device for obese patients. The invention further relates to a medicament delivery device. The invention still further relates to a system comprising a medicament delivery device according and a diameter extension device

### BACKGROUND

Many medicament delivery devices are developed for self-administration, i.e., a user performs the medicament delivery her-, or himself. This requires a medicament delivery device, which is as safe to use and as easy to handle as possible.

In order to meet these requirements, the risk of human errors must be minimized, the number of actions needed to be performed in order to receive a dose need to be reduced and the device must be intuitive and ergonomic to use. Thus, in order to minimize the risk of human errors, it is desirable to have the device as pre-assembled as possible.

Medicament delivery devices, such as autoinjectors and manual injectors, often have a needle shield or needle cover that extends in the proximal direction from the housing. The needle shield protects the needle from exposure before medicament delivery. The needle shield may be arranged to move along the longitudinal axis of the housing in a distal direction further into the housing, when the medicament delivery device is pushed towards an injection site. The needle is thus exposed so that it can penetrate the patient's skin. For autoinjectors, this movement of the needle shield may cause the medicament delivery device to become activated to discharge medicament via the needle.

US 2014/0114247 A1 relates to an injection device comprising a housing, a container holder arranged within said housing, the container holder being configured for accommodating a medicament container having a needle attached to one end thereof and a stopper sealingly and slidable arranged inside the medicament container at the other end thereof, a drive unit comprising a plunger rod and plunger drive means, the plunger drive means being slidable arranged in relation to the plunger rod, being rotationally locked to the plunger rod and being rotatable in relation to the housing.

US 2019/0022334 A1 relates to a safety device for a pre-filled syringe with an injection needle comprising a support body adapted to mount the pre-filled syringe, a needle shield slidably arranged with respect to the support body, a retention and locking means for retaining and locking the needle shield with respect to the support body in a first and a second advanced position and in a retracted position. The needle shield comprises an annular flange adapted to rest on the skin of the patient receiving an injection.

EP 3 007 752 A1 relates to an injection device having a contact surface for pressing against the skin of a patient. The contact surface comprises an inwardly extending or radially extending flange or be at the end of a tapered or flared portion of the sliding sleeve to provide an improved contact area.

U.S. Pat. No. 10,398,854 B2 relates to an injector such as a needle-type autoinjector having a flexible flange disposed at an injection end of the injector for stretching or pinching the skin of the injection site. The flexible flange may be removably attached to the injector or integrated into the injector during manufacturing.

### SUMMARY

Many medicament delivery devices are developed for self-administration. However, if given incorrectly, intramuscular injections may result in poor absorption of drug, reduced drug effectiveness, or irritation to surrounding tissues.

Medicament delivery devices of the types mentioned above, on the market today, do generally not take obese patient skin conditions into account in their designs. For obese patients the amount of subcutaneous tissue present, the weight of the patient, muscle mass, and subcutaneous fat at the injection site may be different. That is, the tissue at the injection site may be softer compared to a normal-weight patient due to the different fat to muscle ratio and thickness of the tissue.

Furthermore, during medicament delivery, the soft skin may fold around the at least part of medicament delivery device and the interaction between the medicament delivery device with the folding skin in obese patients may create friction, which results in that a higher activation force is required.

In other words, a larger force is required to push the needle shield into the housing. Some patients with obesity may suffer from impaired dexterity and/or reduced strength, making it challenging to administer medication.

The above problems contribute to a different resistance of the skin against the activation mechanism of the medicament delivery device. Thus, the medicament delivery device may not be activated correctly.

On the other hand, if more pressure is applied to obtain more resistance for activation, stretching or bunching of the skin during injection is possible.

With the increasing rates of obesity, caution needs to be taken to determine the amount of subcutaneous fat of the tissue at the injection site, to select the correct needle length, to insert at a predefined angle, and to flatten the skin rather than to bunch the skin.

As mentioned above, a medicament delivery device generally comprises a housing body configured to receive and/or hold the main functional components: an activation system, a medicament delivery system, and a drive assembly.

Furthermore, the housing body may be configured to receive a cap to cover a proximal end portion of the medicament delivery device. The cap is usually removed prior to use of the device.

Many examples of a medicament delivery system are known. Examples are a prefilled syringe based system or a cartridge based system. A medicament delivery system generally comprises a delivery member configured to deliver an injection into the tissue. A typical delivery member is a needle.

In order to deliver an injection, the medicament delivery system generally requires energy. Said energy is provided by the drive assembly. Many examples of a drive assembly are known. Generally, a drive assembly comprises a driver and an energy storage member. In some disclosures the energy storage member is referred to as power pack assembly. In one example the energy storage member is a compression or torsion spring; the driver is moved in a proximal direction and coupled to a plunger of a syringe.

Generally, an activation system is configured to activate the injection, i.e. to affect an energy release from the drive assembly to the medicament delivery system. An example the activation system is a push button at the distal end of the housing body configured to activate the drive assembly.

The present disclosure does not provide a solution for the medicament delivery system, activation system, and/or the drive assembly.

An object of the present disclosure is to provide a medicament delivery device which solves, or at least mitigates, problems of the prior art. The above problems are among those solved by the present disclosure as defined in the appended claims.

An embodiment of the present disclosure relates to a diameter extension device for a medicament delivery device, wherein the medicament delivery device comprises: a housing body with a generally tubular structure with a central axis in a proximal-to-distal direction and configured to hold a medicament delivery system and a drive assembly; and a delivery member cover: configured to cover at least a proximal portion of the medicament delivery system, configured to move, when pressed against tissue at an injection site, coaxially with the central axis within the housing body from a first proximal position to at least a second more distal position, and further configured to activate by movement from the first position towards the second position the drive assembly to initiate medicament delivery; and at least two structural features recessing or protruding from an outer surface of the housing body. Wherein the diameter extension device comprises: a body with a generally tubular structure being configured to surround at least a portion of the housing body and to move axially in direction of the central axis on the outer surface of the housing body; and at least two guiding means configured to communicate with the at least two structural features of the housing body in order to define the axial movement of the diameter extension device on the outer surface of the housing body.

An embodiment of the present disclosure relates to diameter extension device for a medicament delivery device, wherein the medicament delivery device comprises: a housing body with a generally tubular structure with a central axis in a proximal-to-distal direction and configured to hold a medicament delivery system and a drive assembly; and a delivery member cover: configured to cover at least a proximal portion of the medicament delivery system, configured to move, when pressed against tissue at an injection site, coaxially with the central axis within the housing body from a first proximal position to at least a second more distal position, and further configured to activate by movement from the first position towards the second position the drive assembly to initiate medicament delivery; and at least two structural features recessing or protruding from an outer surface of the housing body. Wherein the diameter extension device comprises: a body with a generally tubular structure being configured to surround at least a portion of the housing body; at least two grabbing means configured to engage with the at least two structural features in order to hold a distal portion of the diameter extension device fix to the housing body; and a flexible portion configured to allow for axial movement of a proximal portion of the diameter extension device.

In and embodiment of the present disclosure, the diameter extension device further comprises an annular flange at a proximal end portion of the diameter extension device with an outer diameter about the same or larger than an outer diameter of the housing body.

In and embodiment of the present disclosure, the diameter extension device comprises an annular flange at a proximal end portion of the diameter extension device, and the annular flange has a central opening configured to allow the delivery member cover to pass through.

In and embodiment of the present disclosure, the diameter extension device further comprises at least two cap-engagement means configured to engage with a cap of the medicament delivery device.

In and embodiment of the present disclosure, the diameter extension device further comprises at least two distal protrusions; each distal protrusion comprises a slot; and the slot defines the guiding means.

In and embodiment of the present disclosure, the end portions of the slots define a distal stop position and a proximal stop position for the axial movement of the diameter extension device.

In and embodiment of the present disclosure, the cap engagement means are configured to disengage when the diameter extension device is in the proximal stop position.

In and embodiment of the present disclosure, the diameter extension device further comprises at least two flexible arms protruding radially inwardly from the annular flange and configured to engage with the delivery member cover.

In and embodiment of the present disclosure, the flexible arms are configured to be flexed in radially outward and distal direction, when the diameter extension device is in a distal position and/or the cap is attached to the medicament delivery device; the flexible arms are further configured to be relaxed in a radial direction, when the diameter extension device is in the proximal stop position; and the flexible arms are further configured to, when being relaxed at the proximal stop position, to engage with the delivery member cover and to move the delivery member cover from the first to the second position, while the diameter extension device is moved in a distal direction.

In and embodiment of the present disclosure, the diameter extension device comprises at least two pluralities of holes or recesses each arranged in an axial direction and the diameter extension device is configured to engage the pluralities of holes or recesses with the surface feature of the housing body for guiding the axial movement of the diameter extension device.

In and embodiment of the present disclosure, the most proximal of the holes or recesses define a respective distal stopping position of the diameter extension device; and the most distal of the holes or recesses define a respective proximal stopping portion of the diameter extension device.

In and embodiment of the present disclosure, at the proximal stopping position of the diameter extension device the proximal end of the diameter extension device is at the same position or at a further proximal position as the proximal end of the delivery member cover in the first position; and at the distal stopping position of the diameter extension device the proximal end of the diameter extension device is at the same position or at a further distal position as the proximal end of the delivery member cover in the second position.

An embodiment of the present disclosure relates to a medicament delivery device suitable for a diameter extension device according to any one of the preceding embodiments

An embodiment of the present disclosure relates to a system comprising a medicament delivery device according to the preceding embodiment and a diameter extension device according to any one of according to any one of the preceding embodiments.

Based on numerical studies and experimental tests the applicant has realized that the main factor for the optimization of the activation force for obese patients is the ratio between the outer diameter of the housing body and the outer diameter of the needle cover as this factor directly influences the amount of contact between parts of the device and the skin of the injection site.

A further factor is the internal resistance force of the activation mechanism, e.g., the spring force of the needle cover. A lower spring force will reduce the aforementioned problems; however, the optimization is limited because a certain spring force is required to ensure the device functionality as well as to fulfill industry requirements for medicament delivery devices. Thus, in this disclosure this factor is not optimized.

Still a further factor is the offset at full stroke position. The latter being the distance between the contacting surface of the proximal end of the needle cover and the end portion of the housing body. It has been found that increasing this distance lowers the activation force. However, at some offset distance, the activation force on soft tissue will plateau due to less resistance because the soft tissue will have less contact with the housing body. Thus, in this disclosure this factor is not optimized.

It has further been realized that all three factors may be interrelated, however, to a certain extend they can be independently improved.

It is a first general concept of the disclosure, to provide an extended surface area at the injection site, i.e., the proximal end portion of the needle cover. This is illustrated in FIG. 5.

FIG. 5 illustrates the results of a simulation of an injection with different Models of injectors into obese skin 10. In the columns of FIG. 5 results of four different Models of embodiments of the disclosure are shown. The Models differ in the respective amount of diameter extension. In the rows of FIG. 5 two different amounts of force are applied. All figures show results for the same tissue.

Model #1 has no diameter extension and ratio of 0.79. That is, the outer diameter of the needle cover is smaller than the inner diameter of the housing body.

Model #2 has a diameter extension and a ratio of 0.92. Model 3 has a ratio of 1. Model 4 has a ratio of 1.9.

The simulations show that an increased diameter portion has two main effects.

First, it increases the contact surface with the skin 10 and thus reduces the local pressure on the tissue, which allows for more movement in the activation mechanism of the device at the same pressure. This is evident from a comparison at the solid line P1 of the first row. The needle cover of Model #1 has moved the least and the needle cover of Model #4 has moved the most. However, the needle covers of Model #3 and Model #4 have moved almost the same.

Second, the shape of the folding tissue 10 is changed to an extent that the contact between the skin of the injection site and the moving end portion during activation, i.e., needle shield and/or housing body, is reduced or prevented.

This is evident from the second row of FIG. 5 at the solid line P2. The second row shows a subsequent movement of the housing body 1 during activation. While the needle covers in Model #3 and Model #4 have already reached the respective end position, little to none contact between the tissue 10 and on the side surfaces of the housing body 1 is observed. In Contrast the devices according to Model #1 and Model #2 still require more pressure to reach the end position and exhibit a substantial amount of contact between the tissue 10 at the side surfaces of the housing body.

Thus, a ratio of about 1 between the outer diameter of the needle shield 2 and the housing body 1 is a preferred ratio for embodiments of this disclosure. A smaller ratio still allows for some contact with the tissue during activation and a higher ratio does not further optimize the activation force.

The optimum ratio was determined for an essentially flat and circular contact surface with the tissue 10. However, other shapes of contact surfaces are possible. Concave, convex, or complex axial shapes and/or elliptical or complex radial shapes are possible. For said shapes, a comparable ratio is obtained, however, for small deviations from a circular shape the overall effect will be the same.

If the ratio is about 1 and the outer diameter of the housing body is essentially the same as the diameter of the extended surface area portion, the activation force is optimized and the contact with the tissue (except for the intended injection site contact area) is minimized.

It is a second general concept of the disclosure to provide an additional cover, preferably in form of a sleeve, covering a proximal end portion of the needle cover and/or the housing body and thus reduce or prevent the contact with the tissue at the injection site during activation. This is illustrated in FIG. 6.

FIG. 6 shows three different embodiments of the disclosure at the same push pressure. The embodiments on the left, Model #5 and Model #5b are similar to Model #1 and Model #3 as discussed before, respectively. The embodiments of Model #5a has a ratio of about 1. The embodiment Model #6 also has a ratio of about 1, thus it also corresponds to Model #3 as discussed above, but additionally comprises a sleeve portion projecting distally from contact surface. In Model #5a and Model #5b the tissue is in contact with the side surface of the needle cover and the movement of the housing body 1 is not completed, see the two solid lines and the distance Δ therebetween.

In Model #6, at the same pressure, the skin is only in contact with the sleeve portion and the movement is already completed. Thus, the provision of a sleeve portion further optimizes the activation force.

It is a third general inventive concept of this disclosure, to provide a moving or sliding cover, which comes in contact with the tissue, but does reduce the friction, because the skin is not in contact with the activation mechanism.

It is a fourth general concept of this disclosure, that the design of a medicament delivery device, which was originally developed for normal-weight patients, is not changed. Instead, a diameter extension device is provided on the medicament delivery device. The diameter extension device provides the contact surface diameter extension as discussed under the first general concept. The add further provides a sleeve portion, according to the second general concept, and/or a sliding cover, according to the third general concept.

The diameter extension device may be provided as an add-on, as an auxiliary device, and/or as an accessory part. That is, the medicament delivery device may be assembled with the diameter extension device, or the diameter extension device may be provided separately and put on the medicament delivery device prior to use.

In the present disclosure, when the term “distal direction” is used, this refers to the direction pointing away from the dose delivery site during use of the medicament delivery device. When the term “distal part/end” is used, this refers to the part/end of the delivery device, or the parts/ends of the members thereof, which during use of the medicament delivery device is/are located furthest away from the dose delivery site. Correspondingly, when the term “proximal direction” is used, this refers to the direction pointing towards the dose delivery site during use of the medicament delivery device. When the term “proximal part/end” is used, this refers to the part/end of the delivery device, or the parts/ends of the members thereof, which during use of the medicament delivery device is/are located closest to the dose delivery site.

Further, the terms “longitudinal”, “longitudinally”, “axially” and “axial” refer to a direction extending from the proximal end to the distal end and along the device or components thereof, typically in the direction of the longest extension of the device and/or component.

Similarly, the terms “transverse”, “transversal” and “transversally” refer to a direction generally perpendicular to the longitudinal direction.

Further, the terms “circumference”, “circumferential”, or “circumferentially” refer to a circumference or a circumferential direction relative to an axis, typically a central axis extending in the direction of the longest extension of the device and/or component. Similarly, “radial” or “radially” refer to a direction extending radially relative to the axis, and “rotation”, “rotational” and “rotationally” refer to rotation relative to the axis.

Generally, all terms used in the claims are to be interpreted according to their ordinary meaning in the technical field, unless explicitly defined otherwise herein. All references to a/an/the element, apparatus, member, component, means, etc. are to be interpreted openly as referring to at least one instance of the element, apparatus, member component, means, etc., unless explicitly stated otherwise.

### BRIEF DESCRIPTION OF THE DRAWINGS

Embodiments of the present disclosure will now be described by way of example only and with reference to the following accompanying drawings, in which

FIG. 1 shows a perspective view of a medicament delivery device with a diameter extension device according to a first embodiment of the present disclosure.

FIG. 2A shows a cross sectional side view of a medicament delivery device with the diameter extension device and a cap according to the first embodiment of the present disclosure.

FIG. 2B shows a cross sectional side view of a medicament delivery device with the diameter extension device and the cap pulled forward according to the first embodiment of the present disclosure.

FIG. 2C shows a cross sectional side view of a medicament delivery device with the diameter extension device and the cap being removed according to the first embodiment of the present disclosure.

FIG. 2D shows a cross sectional side view of a medicament delivery device with the diameter extension device according to the first embodiment of the present disclosure in a ready to use state.

FIG. 2E shows a cross sectional side view of a medicament delivery device with the diameter extension device according to the first embodiment of the present disclosure after activation.

FIG. 3 shows a side view of a medicament delivery device with a diameter extension device and a RNS remover according to a second embodiment of the present disclosure.

FIG. 4A shows a side view of a medicament delivery device with a diameter extension device and a RNS remover according to the second embodiment of the present disclosure with the RNS remover being pulled in a proximal direction.

FIG. 4B shows a side view of a medicament delivery device with the diameter extension device and a RNS remover according to the second embodiment of the present disclosure with the RNS remover being removed.

FIG. 4C shows a side view of a medicament delivery device with the diameter extension device and a RNS remover according to the second embodiment of the present disclosure with the needle cover and the diameter extension device being flush.

FIG. 4D shows a side view of a medicament delivery device with the diameter extension device and a RNS remover according to the second embodiment of the present disclosure with the needle cover and the diameter extension device being in a distal stop.

FIG. 5 shows results of a numerical simulation of the activation for different embodiments of the present disclosure.

FIG. 6 shows results of a numerical simulation of the activation for different embodiments of the present disclosure.

FIG. 7A shows a perspective view of a diameter extension device according to a third embodiment of the present disclosure.

FIG. 7B shows a perspective view of a medicament delivery device with the diameter extension device according to the third embodiment of the present disclosure and a cap attached.

FIG. 7C shows a perspective view of a medicament delivery device with the diameter extension device according to the third embodiment of the present disclosure and the cap removed.

FIG. 7D shows a perspective view of a medicament delivery device with the diameter extension device according to the third embodiment of the present disclosure in a proximal position.

FIG. 8 shows a perspective view of a diameter extension device according to a fourth embodiment of the present disclosure.

FIG. 9 shows a perspective view of a diameter extension device according to a fifth embodiment of the present disclosure.

FIG. 10 shows a perspective view of a diameter extension device according to a sixth embodiment of the present disclosure.

### DETAILED DESCRIPTION

The inventive concept will now be described more fully hereinafter with reference to the accompanying drawings, in which exemplifying embodiments are shown. The inventive concept may, however, be embodied in many different forms and should not be construed as limited to the embodiments set forth herein; rather, these embodiments are provided by way of example so that this disclosure will be thorough and complete, and will fully convey the scope of the inventive concept to those skilled in the art. Like numbers refer to like members throughout the description.

For the purpose of the disclosure the pre-activation is defined as the button is not pressed, and the used pushes the device against their abdomen first. During pre-activation a force is required to bring the device into the first position.

The medicament delivery device of embodiments according to the present disclosure may comprise a housing 1 configured to house the components of a medicament delivery device. The housing may be tubular. The housing may extend along an axis which is the longitudinal axis of the tubular shaped housing. The housing has a proximal end and a distal end. The housing has a body which extends between the proximal end and the distal end.

The medicament delivery device may further comprise a delivery member cover, also referred to as needle shield. The delivery member cover may be generally tubular or tubular. The delivery member cover is arranged in the housing body and extends proximally from a proximal opening of the housing body.

The delivery member cover may be configured to be moved linearly relative to the housing body along the axis. The delivery member cover is configured to be moved linearly from an extended position to a retracted position relative to the housing body. In the retracted position the delivery member cover is received further by the housing body.

The medicament delivery device may further comprise a drive assembly (not shown) arranged in the housing. The drive assembly may be any drive assembly used in an autoinjector or a manual injector which activates or triggers medicament delivery by movement of the delivery member cover from the extended position towards the retracted position. Examples of suitable drive assemblies are for example disclosed in WO2011/123024, WO2019/011688, WO2019/011689, WO2019/011690, and WO2019/063267, incorporated herein by reference.

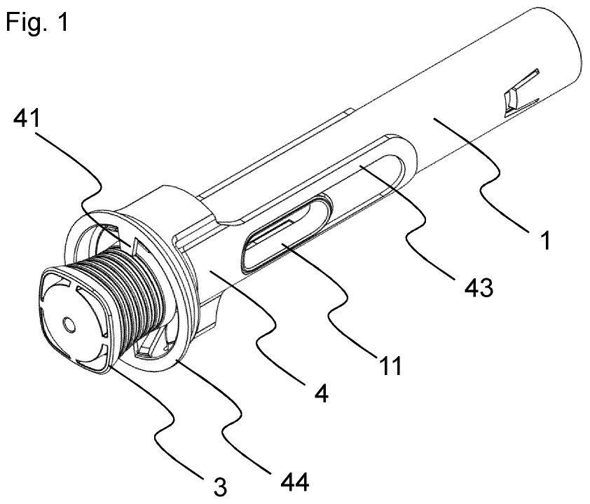

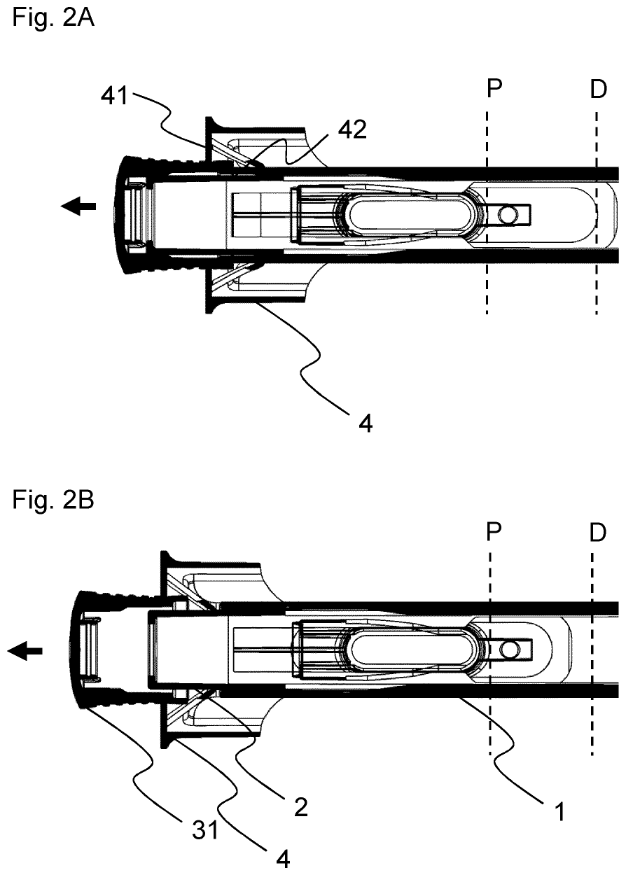

FIG. 1 and FIGS. 2A to 2E illustrates a first embodiment of the present disclosure. FIG. 1 shows a perspective view of a medicament delivery device with a diameter extension device according to a first embodiment of the present disclosure. FIG. 2A shows a cross sectional side view of a medicament delivery device with the diameter extension device and a cap according to the first embodiment of the present disclosure. FIG. 2B shows a cross sectional side view of a medicament delivery device with the diameter extension device and the cap pulled forward according to the first embodiment of the present disclosure. FIG. 2C shows a cross sectional side view of a medicament delivery device with the diameter extension device and the cap being removed according to the first embodiment of the present disclosure. FIG. 2D shows a cross sectional side view of a medicament delivery device with the diameter extension device according to the first embodiment of the present disclosure in a ready to use state. FIG. 2E shows a cross sectional side view of a medicament delivery device with the diameter extension device according to the first embodiment of the present disclosure after activation

According to the first embodiment of the present disclosure a diameter extension device 4 for a medicament delivery device is provided.

In a preferred embodiment, the diameter extension device 4 is of a generally tubular structure and configured to surround at least a proximal end portion of the medicament delivery device.

That is, the diameter extension device 4 is configured to surround at least a proximal end portion of the housing body 1. Furthermore, the diameter extension device also surrounds at least a distal end portion of a cap 3 of the medicament delivery device.

In a preferred embodiment, the diameter extension device is configured to be put on the medicament delivery device after the assembly of the medicament delivery device. Alternatively, the diameter extension device may be configured to be assembled with the medicament delivery device.

The diameter extension device 4 may be integrally formed as one piece. This provides for a very rigid diameter extension device 4. Alternatively, the diameter extension device 4 may be provided in a plurality of pieces, preferably two half-pieces.

In the latter embodiment one or both of the half pieces may comprise linking means, preferably snap-fit means. This provides for a diameter extension device 4, which can be put easily around an existing medicament delivery device prior to use with an obese patient.

In a preferred embodiment, the diameter extension device 4 comprises cap engagement means 41 configured to engage with a structural element of a cap 3 of the medicament delivery device. In a preferred embodiment, the cap engagement means 41 are at least two radial protrusions 41 configured to engage with a structural feature on the surface of the cap 3, preferably with a recess 31 on an outer surface of the cap 3.

The engagement between the cap engagement means 41 and the cap 3 is such that, when the cap 3 is pulled in a proximal direction in order to remove the cap 3, the engagement transfers the force and moves the diameter extension device 4 also in the proximal direction.

In a preferred embodiment, the diameter extension device 4 comprises guiding means 43 configured to guide a movement of the diameter extension device 4 along an outer surface of the housing body 1 based on one or more surface features of the housing body 1.

In a preferred embodiment, the guiding means 43 are or comprises two or more slots configured to engage with a corresponding number of a radially protruding surface features of the outer surface of the housing body 1. Alternatively, the guiding means are or comprises two or more radially inwardly protruding protrusions configured to engage with a corresponding number of radially recessed surface features of the housing body 1.

The housing body surface feature preferably is an axially extended housing body feature. More preferably, the housing body feature is a window 11 of the housing body 1, in particular a protrusion defining a rim or frame of the window 11.

In a preferred embodiment, the surface feature of the housing body 1 defines at least one proximal stopping interface and at least one distal stopping interface for the guiding means 43 to limit the movement of the diameter extension device 4 in a distal and in a proximal direction, respectively.

In a preferred embodiment, when the diameter extension device 4 has moved with the cap 3 into a proximal stopping position, defined by the stopping interfaces and the guiding means 43, the cap 3 and the cap engagement means 41 disengage and the cap 3 is released from the medicament delivery device. The diameter extension device 4 remains on the medicament delivery device.

The distal end of a window 11 may define a distal stopping interface and the proximal end of the window 11 may define a proximal stopping interface, cf. FIG. 1.

In a preferred embodiment, the diameter extension device 4 further comprises an annular flange 44 at a distal end portion of the diameter extension device 4. The cap engagement means 41 may protrude radially inwardly from the annular flange 44. The guiding means 43 may protrude distally from the annular flange 44. The guiding means 43 may be formed as slots in the distal protrusions.

Additionally, but optionally, the diameter extension device 4 may further comprise a sleeve portion protruding distally from the annular flange 44.

The annular flange 44 is configured to reduce or prevent contact between the folding tissue and the medicament delivery device during the activation.

In a preferred embodiment, the diameter extension device 4 further comprises at least two needle shield engagement means 42, see FIG. 2A to 2E. The needle shield engagement means 42 are configured to engage with the needle shield 2, when the diameter extension device 4 is at a proximal stop defined by the guiding means 43.

In a preferred embodiment, the needle shield engagement means 42 are flexible arms 42 protruding radially inwardly from the annular flange 44. When the cap 3 is attached to the device, the flexible arms 42 are flexed in a distal direction. When the cap 3 is removed at the proximal stop, the flexible arms 42 may relax into a position proximal to the end portion of the needle cover 2.

When the device is subsequently pushed in a distal direction the flexible arms 42 may engage with the needle cover 2. During activation of the flexible arms 42, the needle cover 2, and the diameter extension device 4 move in a proximal direction to the proximal stop.

In other words, as shown in FIG. 1, the diameter extension device 4 may comprise an annular flange 44; at least two protrusions protruding distally from the annular flange 44; a slot 43 formed in the respective protrusion configured to surround a window 11 to form guiding means 43; at least two cap engagement means 41 protruding radially inwardly from the annular flange 44 and configured to engage with a recess 31 on a cap 3; and two flexible arms 42 protruding radially inwardly from the annular flange 44 configured to engage with the needle cover 2, when the diameter extension device 4 is in a proximal stop position.

The slot 43 is axially longer than the window 11 and a proximal end of the window 11 thus defines a distal stop for the diameter extension device movement and the distal end of the window 11 thus defines a proximal stop for the diameter extension device 4, as will be discussed below and is illustrated in FIGS. 2A to 2E.

In FIGS. 2A to 2E the movements during pre-activation and activation are shown for the first embodiment of the present disclosure.

In FIG. 2A, the diameter extension device 4 is in the distal stop position. The distal stop is defined by the distal stopping interface. That is, the proximal end of the window 11 contacting a proximal end of the slot 43 and thus limits the movement of the diameter extension device 4 in a most distal position. The position is indicated by D and a dashed line.

The cap 3 is attached and the cap engagement means 41 are engaged with a recess 31 of the cap 3, preferably a recess 31 at the distal end of the cap 3. The flexible arms 42 are in a distally flexed position and may contact the needle cover 2 or the housing body 1.

In order to remove the cap 3, a user pulls the cap 3 in a proximal direction.

In FIG. 2B, the cap is pulled in a proximal direction, as indicated by the arrow. The diameter extension device 4 has moved with the cap 3 in a proximal direction, because of the force transfer via the cap engagement means 41. The slot 43 as moved accordingly. The window 11 and the slot 43 guide the movement of the diameter extension device 4. The cap 3 and/or the needle cover 2 block the flexible arms 42 from relaxing into a proximal position.

The user continues to pull the cap 3 until it is released from the diameter extension device 4. The cap 3 is released, when the diameter extension device is in the proximal stop and the cap engagement means 41 disengage. Subsequently the user may push the diameter extension device 4 back in a distal direction to activate the device. This pushing may be realized by placing the diameter extension device 4 and device against the tissue of an injection site and pushing the device in a proximal direction against the injection site, which moves the diameter extension device 4 in a distal direction.

In FIG. 2C, the diameter extension device 4 is shown shortly after or at the proximal stop position with the cap 3 being already removed. The proximal stop is defined by the proximal stopping interface. That is, the distal end of the window 11 contacts the distal end of the slot 43 and thus limits the movement of the diameter extension device 4 in a proximal direction. The position is indicated by P and a dashed line.

As discussed above, at the proximal stop the cap 3 is disengaged. Furthermore, the free-ends of the flexible arms 42 have passed a distal end portion of the needle cover 2. The flexible arms 42 can now relax in to a radially inwardly and proximal direction, as indicated by the bent arrows.

When the flexible arms 42 are in the relaxed position they may engage with the needle cover 2. Preferably, the flexible arms 42 contact a proximal end surface of the needle cover 2. The flexible arms 42, the annular flange 44, and the cap engagement means 41 form together with the proximal end surface of the needle cover 2 a contact surface.

The contact surface has an enlarged diameter and thus reduces or prevents contact with the folding tissue. Thus, the activation force is reduced.

In FIG. 2D, the diameter extension device 4 is pushed in a distal direction, i.e., the housing body 1 is pushed in a proximal direction against the injection site. That is, the diameter extension device 4 moves proximally from P to D, the diameter extension device 4 pushes the needle cover 2 proximally. Thus, the activation movement is initiated.

In FIG. 2E, the diameter extension device 4 is in the distal stop again. The needle cover 2 has been moved into a distal position and the medicament delivery device has been activated.

The diameter extension device may be configured to be removed from the medicament delivery device and to be reused.

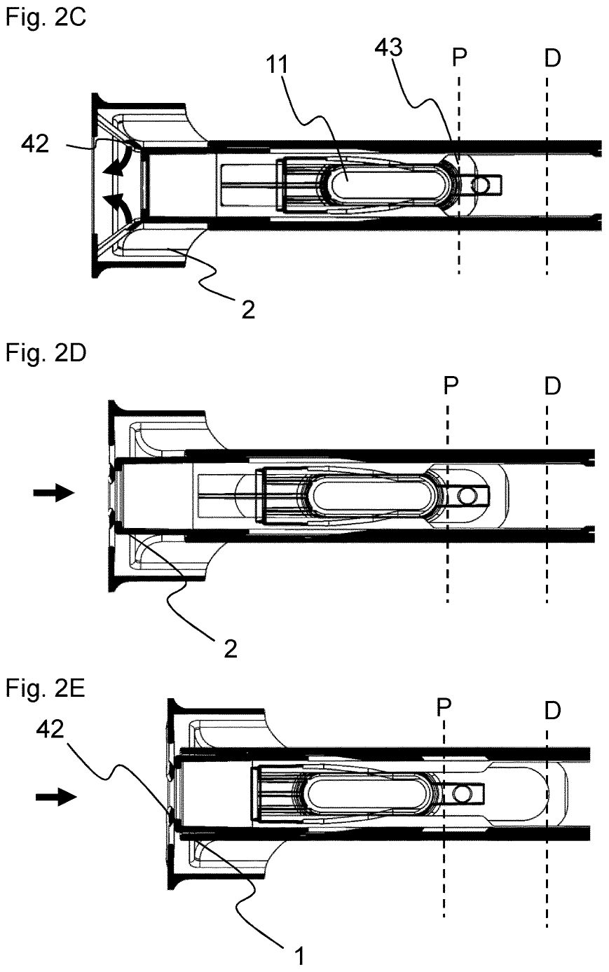

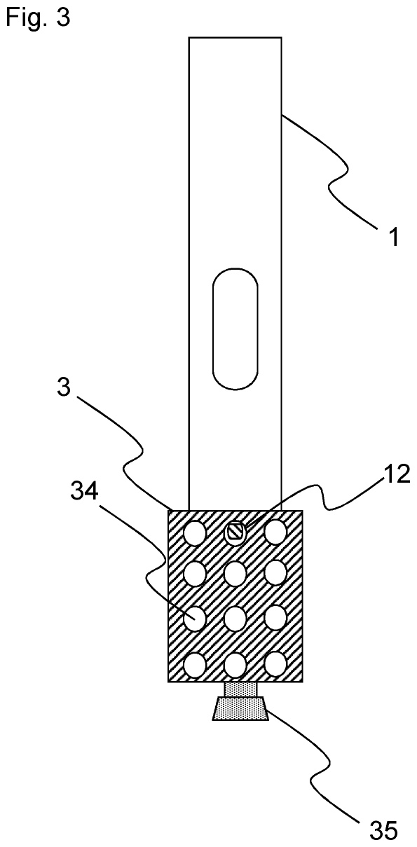

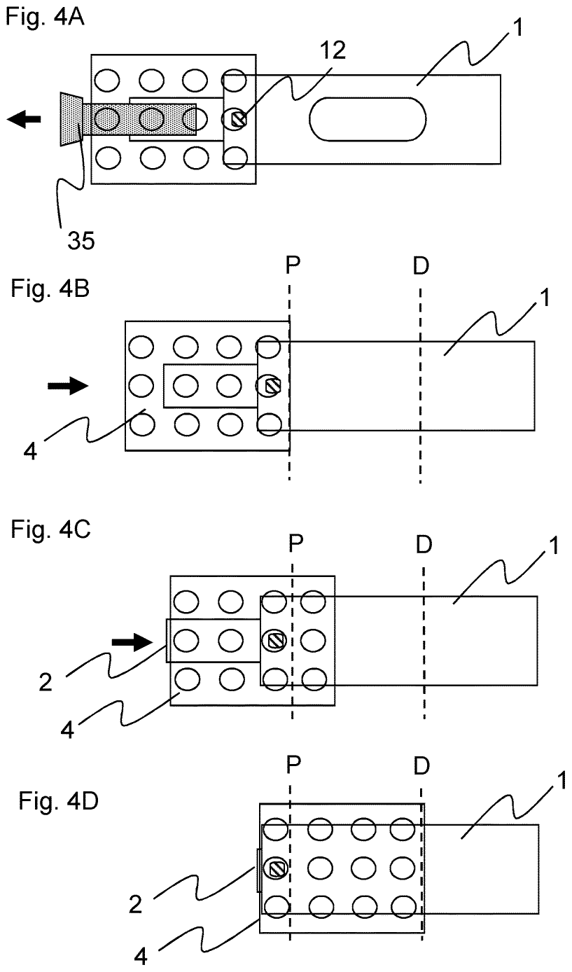

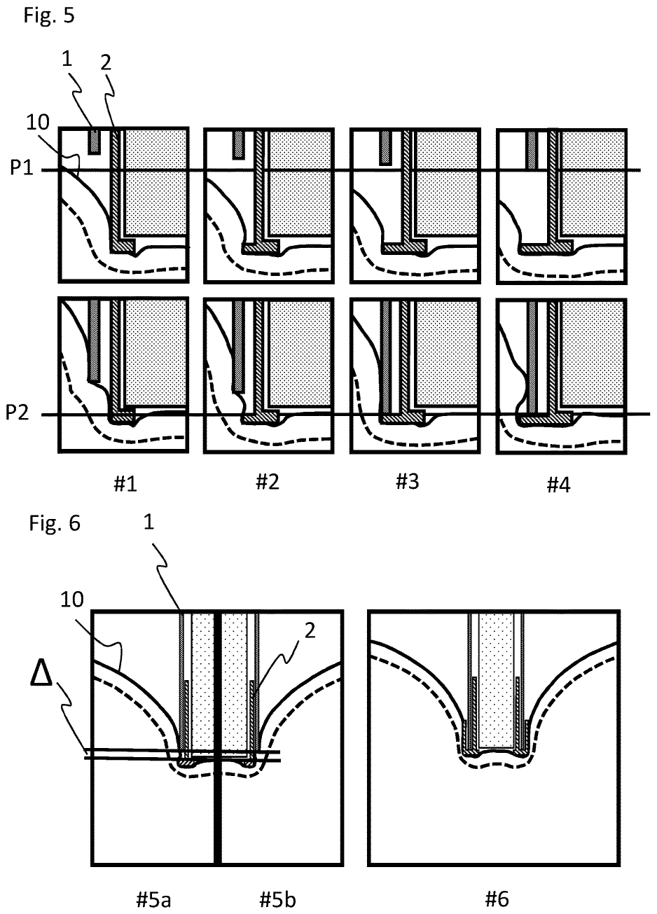

FIG. 3 and FIGS. 4A to 4D illustrate a second embodiment according to the present disclosure. FIG. 3 shows a side view of a medicament delivery device with a diameter extension device and a RNS remover according to a second embodiment of the present disclosure. FIG. 4A shows a side view of a medicament delivery device with a diameter extension device and a RNS remover according to the second embodiment of the present disclosure with the RNS remover being pulled in a proximal direction. FIG. 4B shows a side view of a medicament delivery device with the diameter extension device and a RNS remover according to the second embodiment of the present disclosure with the RNS remover being removed. FIG. 4C shows a side view of a medicament delivery device with the diameter extension device and a RNS remover according to the second embodiment of the present disclosure with the needle cover and the diameter extension device being flush. FIG. 4D shows a side view of a medicament delivery device with the diameter extension device and a RNS remover according to the second embodiment of the present disclosure with the needle cover and the diameter extension device being in a distal stop.

In the second embodiment the diameter extension device is of tubular structure. The surface features of the housing body are preferably two or more knobs 12 of the housing body 1 configured to hold a cap (not shown) to the medicament delivery device. The guiding means are a series of holes 34 in the diameter extension device 4 configured to engage with the knobs 12.

In a preferred embodiment, when the diameter extension device 4 is attached to the medicament delivery device, the needle cover 2 is secured by a separate rigid needle shield, RNS, remover 35. With cap removed and replaced by the diameter extension device and the RNS remover, as shown in FIG. 3, the medicament delivery device can be securely stored until further use.

In a preferred embodiment, the diameter extension device 4 comprises, two or more pluralities of holes 34 arranged in an axial direction and at positions corresponding to the knobs 12 on the outer surface of the housing body. The last distal hole may define a proximal stop of the diameter extension device and the last proximal hole may define a distal stop of the diameter extension device 4, as illustrated in FIGS. 4A to 4D.

The proximal end portion of the diameter extension device 4 may optionally comprise an annular flange. The proximal end portion of the diameter extension device 4 and the needle cover 2 define a contact area with the tissue of an injection site, see FIG. 4C.

The contact area has an enlarged diameter with a ratio of about 1 and also forms a moving cover, and thus prevents contact between the folding tissue and the moving needle cover and housing body according to the four general concepts of the disclosure.

In FIG. 4A, the diameter extension device 4 and the RNS remover 35 are attached to the medicament delivery device. The diameter extension device 4 in in the proximal stop defined by the most distal one of the pluralities of holes 34. The RNS remover 35 may be removed by pulling it in the proximal direction, as indicated by the arrow. The proximal stop is indicated by P and a dashed line.

In FIG. 4B, the RNS remover 35 has been removed and the diameter extension device 4 is pushed in a distal direction, as indicated by the arrow. As discussed above, the medicament delivery device may be pushed in a proximal direction against the tissue of an injection site.

In FIG. 4C, the add-on 4 is moving in a distal direction on the outer surface of the housing body 1. Accordingly, the holes 34 and the knob 12 move in counter directional, that is, depending on the perspective, the knob 12 has moved from the most distal to a more proximal one of the pluralities of holes 34. The proximal end portion of the needle cover 2 may now become flush with the proximal end of the diameter extension device 4, and activation may start.

The number of holes in the Figures is solely for illustration and not intended limiting. Also, the activation may start already, while the knob is still in the most proximal hole.

In FIG. 4D, the diameter extension device is in the distal stop. That is the movement of the diameter extension device 4 has been completed and the knob 12 is in the most proximal one of the holes 34. The distal stop is indicated by D and a dashed line.

While in the above a plurality of radially directed through holes have been discussed, the disclosure is not limited thereto. Alternatively, the holes may be provided on the inner surface of diameter extension device in form of recesses.

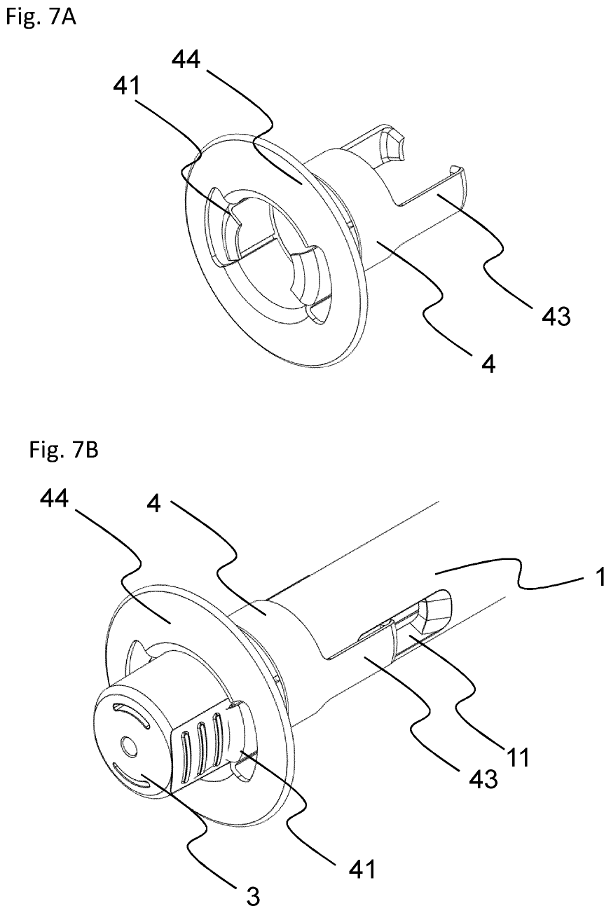

FIGS. 7A to 7D illustrate a third embodiment according to the present disclosure. FIG. 7A shows a perspective view of a diameter extension device according to a third embodiment of the present disclosure. FIG. 7B shows a perspective view of a medicament delivery device with the diameter extension device according to the third embodiment of the present disclosure and a cap attached. FIG. 7C shows a perspective view of a medicament delivery device with the diameter extension device according to the third embodiment of the present disclosure and the cap removed. FIG. 7D shows a perspective view of a medicament delivery device with the diameter extension device according to the third embodiment of the present disclosure in a proximal position.

The third embodiment is based on the first embodiment. The third embodiment differs in the different structural formation of the cap engagement means and the guiding means, however, the functional interplay of said elements remains the essentially the same.

The guiding means 43 are preferably formed as two or more distal protrusions of the diameter extension device 4 each with a radially inwardly protruding distal end portion.

The guiding means are configured to interplay with at least two visualization windows 11 of the housing body to guide and limit the movement of the diameter extension device 4. This is shown in FIG. 7B.

The distally protruding portion of the guiding means 43 may preferably be configured to slide on an outer surface of the housing body 1 and thus form a guide area for the diameter extension device 4 on the housing body 1.

The respective distal end portions are configured to be inserted, preferably to snap into, a respective window 11 of the housing body 1. That is, the distal end portions are configured to move within the window 11.

Thus, the respective distal end of the window forms a distal stopping surface for the distal movement for the diameter extension device 4 and the proximal end of the window forms a proximal stopping surface for the diameter extension device 4.

Additionally or alternatively, instead of the windows 11 one or more structural elements on the outer surface of the housing body may serve to guide and limit the movement of the diameter extension device 4 via interaction with the distal end portions.

The can engagement means 41, also referred to as snappers, are formed as two or more proximal protrusions of the diameter extension device 4 each with a radially inwardly protruding proximal end portion. This is shown in FIG. 7A.

Preferably, the end proximal end portions are configured to engage with at least one structural element on an outer surface of a cap 3 and with at least one structural element on the proximal end surface of the needle cover 2.

Preferably, the cap engagement means are formed as snappers 41. Preferably, the at least one structural element on an outer surface of a cap 3 is a recess on the surface of the cap 3.

FIG. 7B is showing the diameter extension device 4 being attached to the medicament delivery device with the cap 3 attached. In this configuration, the medicament delivery device can be stored and/or delivered to the user. The operation of the medicament delivery device with the diameter extension device for the user is essentially the same as without.

In order to prepare the device for injection, the user may remove the cap 3 by pulling the cap 3 in a proximal direction. The diameter extension device 4 is engaged with the cap 3 via the cap engagement means 41. The diameter extension device 4 thus follows the movement of the cap 3.

When the distal end portions of the guiding means contact the proximal stopping surface, the proximal movement of the diameter extension device 4 is stopped. This is shown in FIG. 7C.

At this position the further movement of the cap 3 results in a disengagement of the cap engagement means and a release from the cap 3.

In a preferred embodiment, the cap engagement means are configured to engage with the proximal end surface of the needle cover 2 when the cap is not attached. This is shown in FIG. 7D.

In other words, when the cap 3 is attached, the snappers 41 are flexed radially outward to provide an inward directed holding force to engage with the cap. When the cap 3 is not attached the snappers 41 are relaxed radially inwardly such that the proximal end portions overlap with the needle cover.

The diameter extension device 4 further comprises an annular flange portion 44 as described above.

In a preferred embodiment, the proximal end portions form a contact surface for the injection site with the annular flange. That is, the proximal end portions preferably do not proximally protrude further than the annular flange.

During the activation movement, i.e., while the user pushes the device in a proximal direction against the injection site, the diameter extension device moves distally with the needle cover and prevents contact with the skin of the injection site as discussed above.

The utilization of the accessory diameter extension device of any of the above embodiments does not require any modification of the medicament delivery device, nor does it require a change of the sequence of use of the device. The accessory is described to be used with an autoinjector with a visualization window, but it can also be utilized in the same way with different autoinjectors.

The needle penetration depth may be reduced by using this accessory, based on the position of the stopping surfaces.

In a preferred embodiment the interaction between the guiding means 43 and the window 11 holds the medicament delivery device and the diameter extension device 4 together and limits the proximal-to-distal displacement of the diameter extension device 4 in respect to the medicament delivery device.

Preferably, the view through the visualization window is not blocked.

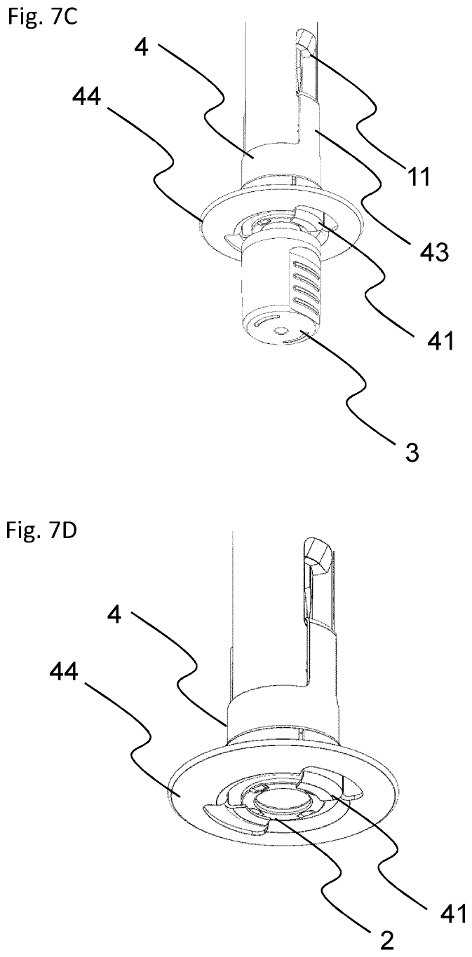

FIG. 8 to FIG. 10 show a fourth, fifth, and sixth embodiment of the present disclosure, which share the general inventive concept, that the diameter extension device may be fixed to the housing body of a medicament delivery device and a flexible portion of the diameter extension device allows for sufficient axial movement of the extended diameter portion.

As in the above-described embodiments, the fourth, fifth, and sixth embodiment of the present disclosure are based on the principle that increasing the area of contact of the device against the skin eases the activation of the device.

Furthermore, the utilization of the accessory diameter extension device does not require any modification of the medicament delivery device parts, nor of the sequence of use of the medicament delivery device. Needle penetration depth may be reduced by using this accessory.

The accessory diameter extension device is a device that is mounted on the autoinjector and that presents three main features: grabbing means, strain relief sleeve, i.e., a compression area, and annular flange portion.

The accessory diameter extension device is hold to the autoinjector by the grabbing means, which is preferably attached to the two visualization windows of the housing body. This holding maintains both devices together and blocks the vertical displacement of the accessory diameter extension device in respect to the medicament delivery device, while does not block the visualization window.

The accessory diameter extension device further comprises two snappers that attach to the medicament delivery device at different points, and which limit the extension of the accessory diameter extension device, i.e., the extension of the strain relief sleeve part.

The middle part of the accessory diameter extension device is the strain relief sleeve. This behaves as a plastic spring, with the capability to extend and to be compressed. With this extension/compression, the annular flange portion of the device is moved axially during the sequence of use of the medicament delivery device.

The accessory diameter extension device has three main positions:
1. Initial position—compression

This is the position of the accessory diameter extension device in the packaging and during storage. The user will receive the device at this position. The extension of the strain relief sleeve is limited by the two snappers, which are radially pressing the needle cover, held inside a gap between a front of the housing body and a cap. The strain relief sleeve (plastic spring) area is under compression. The force of the plastic spring under compression is lower than the force required to remove the cap of the medicament delivery device.

When the user wants to use the device, they remove the cap, and the strain relief sleeve area becomes uncompressed and extends.
2. Removed cap

This is the position after cap removal and during the injection of the device, while in contact with the skin. The strain relief sleeve has extended until the two snappers have moved in a position proximal to the needle cover.

The increased surface is in contact with the skin and moves together with the needle cover dur to the engagement of the two snappers.
3. End of use

Maximum extension occurs at the end of use. Once the user has performed the injection, the needle cover of the medicament delivery device extends. The accessory diameter extension device is then pushed, and it moves together with the needle cover extension.

After that, both the autoinjector and the accessory can be discarded together.

FIG. 8 shows a perspective view of a diameter extension device according to a fourth embodiment of the present disclosure. The diameter extension device is based on the third embodiment as described above. The above discussed guiding means, however, have been replaced by grabbing means 45 configured to hold the diameter extension device 4 to a surface feature of the housing body 1, preferably to one or more visualization windows 11 of the housing body 1.

The diameter extension device further comprises a flexible means, also referred to as strain relief sleeve, 46. In a preferred embodiment the strain relief sleeve 46 achieves its spring functionality by modifying the thickness of stripes of this portion.

That is, a thinner wall of a certain material could be more easily compressed than a thicker wall area. Rigid materials need to be avoided for this solution.

In this case, higher stretchability could be achieved by making stripes that cover all 360° around the accessory.

The spring functionality can also be achieved by combining two different materials: a stretchable one and a rigid one.

The function of the snappers 41 and the annular flange portion 44 is essentially the same as discussed above.

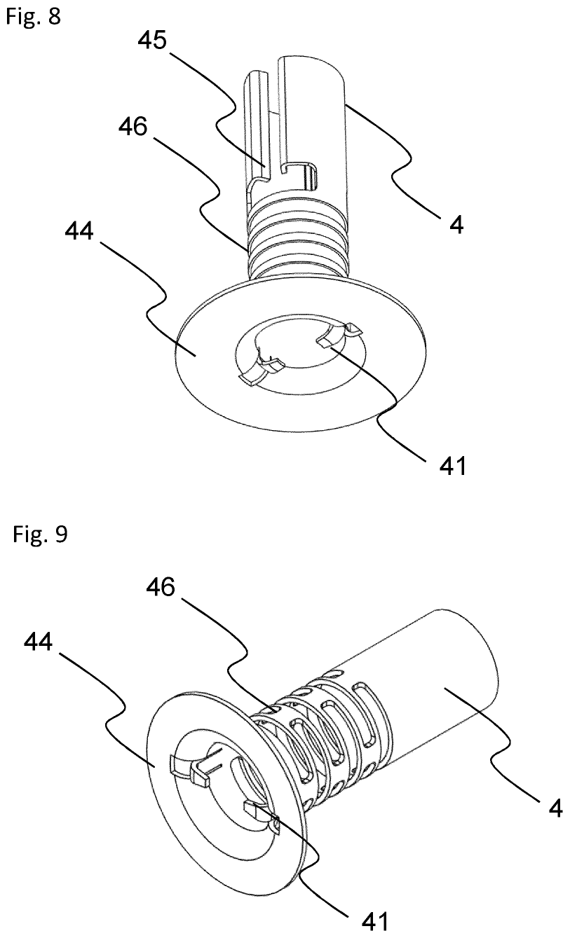

FIG. 9 shows a perspective view of a diameter extension device according to a fifth embodiment of the present disclosure. This embodiment is based on the third and fourth embodiment as discussed above.

The grabbing means are not shown in the drawings and can be any of the grabbing means discussed above.

The strain relief sleeve 46 is perforated at different levels, which gives the accessory diameter extension device a certain compression rate. The holes are horizontally located and 90° tilted alternatingly.

The number of holes and the dimensions are selected based on the required flexibility and the material properties of the diameter extension device.

The function of the snappers 41 and the annular flange portion 44 is essentially the same as discussed above.

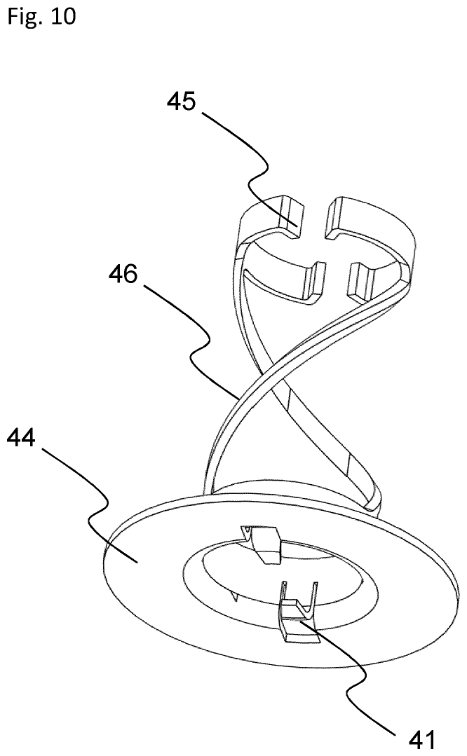

FIG. 10 shows a perspective view of a diameter extension device according to a sixth embodiment of the present disclosure.

In this alternative, the spring behaviour is achieved by a helix spring 46. In this embodiment a rotation effect intrinsic to this type of spring may occur. This effect may be mitigated by changing the compression states in the states discussed above. Instead of the above compression scheme, the middle extension could be a relaxed position and then the device could be further extended by stretching the spring area. This was only half the rotation is observed.

The above concepts may be modified by replacing the strain relief sleeve 46 by any sort of flexible portion, in particular a spring portion formed in the strain relief sleeve 46 may have any spring shape known to the skilled person.

The delivery devices described herein can be used for the treatment and/or prophylaxis of one or more of many different types of disorders.

Exemplary disorders include, but are not limited to: rheumatoid arthritis, inflammatory bowel diseases (e.g. Crohn's disease and ulcerative colitis), hypercholesterolaemia and/or dyslipidemia, cardiovascular disease, diabetes (e.g. type 1 or 2 diabetes), psoriasis, psoriatic arthritis, spondyloarthritis, hidradenitis suppurativa, Sjogren's syndrome, migraine, cluster headache, multiple sclerosis, neuromyelitis optica spectrum disorder, anaemia, thalassemia, paroxysmal nocturnal hemoglobinuria, hemolytic anaemia, hereditary angioedema, systemic lupus erythematosus, lupus nephritis, myasthenia gravis, Behcet's disease, hemophagocytic lymphohistiocytosis, atopic dermatitis, retinal diseases (e.g., age-related macular degeneration, diabetic macular edema), uveitis, infectious diseases, bone diseases (e.g., osteoporosis, osteopenia), asthma, chronic obstructive pulmonary disease, thyroid eye disease, nasal polyps, transplant, acute hypoglycaemia, obesity, anaphylaxis, allergies, sickle cell disease, Alzheimer's disease, Parkinson's disease, dementia with Lewy bodies, systemic infusion reactions, immunoglobulin E (IgE)-mediated hypersensitivity reactions, cytokine release syndrome, immune deficiencies (e.g., primary immunodeficiency, chronic inflammatory demyelinating polyneuropathy), enzyme deficiencies (e.g., Pompe disease, Fabry disease, Gaucher disease), growth factor deficiencies, hormone deficiencies, coagulation disorders (e.g., hemophilia, von Willebrand disease, Factor V Leiden), and cancer.

Exemplary types of drugs that could be included in the delivery devices described herein include, but are not limited to, small molecules, hormones, cytokines, blood products, enzymes, vaccines, anticoagulants, immunosuppressants, antibodies, antibody-drug conjugates, neutralizing antibodies, reversal agents, radioligand therapies, radioisotopes and/or nuclear medicines, diagnostic agents, bispecific antibodies, proteins, fusion proteins, peptibodies, polypeptides, pegylated proteins, protein fragments, nucleotides, protein analogues, protein variants, protein precursors, protein derivatives, chimeric antigen receptor T cell therapies, cell or gene therapies, oncolytic viruses, or immunotherapies.

Exemplary drugs that could be included in the delivery devices described herein include, but are not limited to, immuno-oncology or bio-oncology medications such as immune checkpoints, cytokines, chemokines, clusters of differentiation, interleukins, integrins, growth factors, coagulation factors, enzymes, enzyme inhibitors, retinoids, steroids, signaling proteins, pro-apoptotic proteins, anti-apoptotic proteins, T-cell receptors, B-cell receptors, or costimulatory proteins.

Exemplary drugs that could be included in the delivery devices described herein include, but are not limited to, those exhibiting a proposed mechanism of action, such as human epidermal growth factor receptor 2 (HER-2) receptor modulators, interleukin (IL) modulators, interferon (IFN) modulators, complement modulators, glucagon-like peptide-1 (GLP-1) modulators, glucose-dependent insulinotropic polypeptide (GIP) modulators, cluster of differentiation 38 (CD38) modulators, cluster of differentiation 22 (CD22) modulators, C1 esterase modulators, bradykinin modulators, C-C chemokine receptor type 4 (CCR4) modulators, vascular endothelial growth factor (VEGF) modulators, B-cell activating factor (BAFF), P-selectin modulators, neonatal Fc receptor (FcRn) modulators, calcitonin gene-related peptide (CGRP) modulators, epidermal growth factor receptor (EGFR) modulators, cluster of differentiation 79B (CD79B) modulators, tumor-associated calcium signal transducer 2 (Trop-2) modulators, cluster of differentiation 52 (CD52) modulators, B-cell maturation antigen (BCMA) modulators, enzyme modulators, platelet-derived growth factor receptor A (PDGFRA) modulators, cluster of differentiation 319 (CD319 or SLAMF7) modulators, programmed cell death protein 1 and programmed death-ligand 1 (PD-1/PD-L1) inhibitors/modulators, B-lymphocyte antigen cluster of differentiation 19 (CD19) inhibitors, B-lymphocyte antigen cluster of differentiation 20 (CD20) modulators, cluster of differentiation 3 (CD3) modulators, cytotoxic T-lymphocyte-associated protein 4 (CTLA-4) inhibitors, T-cell immunoglobulin and mucin-domain containing-3 (TIM-3) modulators, T cell immunoreceptor with Ig and ITIM domains (TIGIT) modulators, V-domain Ig suppressor of T cell activation (VISTA) modulators, indoleamine 2,3-dioxygenase (IDO or INDO) modulators, poliovirus receptor-related immunoglobulin domain-containing protein (PVRIG) modulators, lymphocyte-activation gene 3 (LAG3; also known as cluster of differentiation 223 or CD223) antagonists, cluster of differentiation 276 (CD276 or B7-H3) antigen modulators, cluster of differentiation 47 (CD47) antagonists, cluster of differentiation 30 (CD30) modulators, cluster of differentiation 73 (CD73) modulators, cluster of differentiation 66 (CD66) modulators, cluster of differentiation w137 (CDw137) agonists, cluster of differentiation 158 (CD158) modulators, cluster of differentiation 27 (CD27) modulators, cluster of differentiation 58 (CD58) modulators, cluster of differentiation 80 (CD80) modulators, cluster of differentiation 33 (CD33) modulators, cluster of differentiation 159 (CD159 or NKG2) modulators, glucocorticoid-induced TNFR-related (GITR) protein modulators, Killer Ig-like receptor (KIR) modulators, growth arrest-specific protein 6 (GAS6)/AXL pathway modulators, A proliferation-inducing ligand (APRIL) receptor modulators, human leukocyte antigen (HLA) modulators, epidermal growth factor receptor (EGFR) modulators, B-lymphocyte cell adhesion molecule modulators, cluster of differentiation w123 (CDw123) modulators, Erbb2 tyrosine kinase receptor modulators, endoglin modulators, mucin modulators, mesothelin modulators, hepatitis A virus cellular receptor 2 (HAVCR2) antagonists, cancer-testis antigen (CTA) modulators, tumor necrosis factor receptor superfamily, member 4 (TNFRSF4 or OX40) modulators, adenosine receptor modulators, inducible T cell co-stimulator (ICOS) modulators, cluster of differentiation 40 (CD40) modulators, tumor-infiltrating lymphocytes (TIL) therapies, or T-cell receptor (TCR) therapies.

Exemplary drugs that could be included in the delivery devices described herein include, but are not limited to: etanercept, abatacept, adalimumab, evolocumab, exenatide, secukinumab, erenumab, galcanezumab, fremanezumab-vfrm, alirocumab, methotrexate (amethopterin), tocilizumab, interferon beta-1a, interferon beta-1b, peginterferon beta-1a, sumatriptan, darbepoetin alfa, belimumab, sarilumab, semaglutide, dupilumab, reslizumab, omalizumab, glucagon, epinephrine, naloxone, insulin, amylin, vedolizumab, eculizumab, ravulizumab, crizanlizumab-tmca, certolizumab pegol, satralizumab, denosumab, romosozumab, benralizumab, emicizumab, tildrakizumab, ocrelizumab, ofatumumab, natalizumab, mepolizumab, risankizumab-rzaa, ixekizumab, and immune globulins.

Exemplary drugs that could be included in the delivery devices described herein may also include, but are not limited to, oncology treatments such as ipilimumab, nivolumab, pembrolizumab, atezolizumab, durvalumab, avelumab, cemiplimab, rituximab, trastuzumab, ado-trastuzumab emtansine, fam-trastuzumab deruxtecan-nxki, pertuzumab, transtuzumab-pertuzumab, alemtuzumab, belantamab mafodotin-blmf, bevacizumab, blinatumomab, brentuximab vedotin, cetuximab, daratumumab, elotuzumab, gemtuzumab ozogamicin, 90-Yttrium-ibritumomab tiuxetan, isatuximab, mogamulizumab, moxetumomab pasudotox, obinutuzumab, ofatumumab, olaratumab, panitumumab, polatuzumab vedotin, ramucirumab, sacituzumab govitecan, tafasitamab, or margetuximab.

Exemplary drugs that could be included in the delivery devices described herein include “generic” or biosimilar equivalents of any of the foregoing, and the foregoing molecular names should not be construed as limiting to the “innovator” or “branded” version of each, as in the non-limiting example of innovator medicament adalimumab and biosimilars such as adalimumab-afzb, adalimumab-atto, adalimumab-adbm, and adalimumab-adaz.

Exemplary drugs that could be included in the delivery devices described herein also include, but are not limited to, those used for adjuvant or neoadjuvant chemotherapy, such as an alkylating agent, plant alkaloid, antitumor antibiotic, antimetabolite, or topoisomerase inhibitor, enzyme, retinoid, or corticosteroid. Exemplary chemotherapy drugs include, by way of example but not limitation, 5-fluorouracil, cisplatin, carboplatin, oxaliplatin, doxorubicin, daunorubicin, idarubicin, epirubicin, paclitaxel, docetaxel, cyclophosphamide, ifosfamide, azacitidine, decitabine, bendamustine, bleomycin, bortezomib, busulfan, cabazitaxel, carmustine, cladribine, cytarabine, dacarbazine, etoposide, fludarabine, gemcitabine, irinotecan, leucovorin, melphalan, methotrexate, pemetrexed, mitomycin, mitoxantrone, temsirolimus, topotecan, valrubicin, vincristine, vinblastine, or vinorelbine.

Exemplary drugs that could be included in the delivery devices described herein also include, but are not limited to, analgesics (e.g., acetaminophen), antipyretics, corticosteroids (e.g. hydrocortisone, dexamethasone, or methylprednisolone), antihistamines (e.g., diphenhydramine or famotidine), antiemetics (e.g., ondansetron), antibiotics, antiseptics, anticoagulants, fibrinolytics (e.g., recombinant tissue plasminogen activator [r-TPA]), antithrombolytics, or diluents such as sterile water for injection (SWFI), 0.9% Normal Saline, 0.45% normal saline, 5% dextrose in water, 5% dextrose in 0.45% normal saline, Lactated Ringer's solution, Heparin Lock Flush solution, 100 U/mL Heparin Lock Flush Solution, or 5000 U/mL Heparin Lock Flush Solution.

Pharmaceutical formulations including, but not limited to, any drug described herein are also contemplated for use in the delivery devices described herein, for example pharmaceutical formulations comprising a drug as listed herein (or a pharmaceutically acceptable salt of the drug) and a pharmaceutically acceptable carrier. Such formulations may include one or more other active ingredients (e.g., as a combination of one or more active drugs), or may be the only active ingredient present, and may also include separately administered or co-formulated dispersion enhancers (e.g., an animal-derived, human-derived, or recombinant hyaluronidase enzyme), concentration modifiers or enhancers, stabilizers, buffers, or other excipients.

Exemplary drugs that could be included in the delivery devices described herein include, but are not limited to, a multi-medication treatment regimen such as AC, Dose-Dense AC, TCH, GT, EC, TAC, TC, TCHP, CMF, FOLFOX, mFOLFOX6, mFOLFOX7, FOLFCIS, CapeOx, FLOT, DCF, FOLFIRI, FOLFIRINOX, FOLFOXIRI, IROX, CHOP, R-CHOP, RCHOP-21, Mini-CHOP, Maxi-CHOP, VR-CAP, Dose-Dense CHOP, EPOCH, Dose-Adjusted EPOCH, R-EPOCH, CODOX-M, IVAC, HyperCVAD, R-HyperCVAD, SC-EPOCH-RR, DHAP, ESHAP, GDP, ICE, MINE, CEPP, CDOP, GemOx, CEOP, CEPP, CHOEP, CHP, GCVP, DHAX, CALGB 8811, HIDAC, MopAD, 7+3, 5+2, 7+4, MEC, CVP, RBAC500, DHA-Cis, DHA-Ca, DHA-Ox, RCVP, RCEPP, RCEOP, CMV, DDMVAC, GemFLP, ITP, VIDE, VDC, VAI, VDC-IE, MAP, PCV, FCR, FR, PCR, HDMP, OFAR, EMA/CO, EMA/EP, EP/EMA, TP/TE, BEP, TIP, VIP, TPEx, ABVD, BEACOPP, AVD, Mini-BEAM, IGEV, C-MOPP, GCD, GEMOX, CAV, DT-PACE, VTD-PACE, DCEP, ATG, VAC, VeIP, OFF, GTX, CAV, AD, MAID, AIM, VAC-IE, ADOC, or PE.

Various modifications to the embodiments described are possible and will occur to those skilled in the art without departing from the invention which is defined by the following claims.

1-15. (canceled)

16. A diameter extension device for a medicament delivery device, wherein the medicament delivery device comprises:
a housing body with a generally tubular structure with a central axis in a proximal-to-distal direction and configured to hold a medicament delivery system and a drive assembly; and
a delivery member cover:
configured to cover at least a proximal portion of the medicament delivery system,
configured to move, when pressed against tissue at an injection site, coaxially with the central axis within the housing body from a first proximal position to at least a second more distal position, and
further configured to activate by movement from the first position towards the second position the drive assembly to initiate medicament delivery; and
at least two structural features recessing or protruding from an outer surface of the housing body; and
wherein the diameter extension device comprises:
a body with a generally tubular structure being configured to surround at least a portion of the housing body and to move axially in direction of the central axis on the outer surface of the housing body; and
at least two guiding means configured to communicate with the at least two structural features of the housing body in order to define the axial movement of the diameter extension device on the outer surface of the housing body.

17. The diameter extension device according to claim 16,
wherein the diameter extension device further comprises an annular flange at a proximal end portion of the diameter extension device with an outer diameter about the same or larger than an outer diameter of the housing body.

18. The diameter extension device according to claim 16,
wherein the diameter extension device comprises an annular flange at a proximal end portion of the diameter extension device, and
wherein the annular flange has a central opening configured to allow the delivery member cover to pass through.

19. The diameter extension device according to claim 16,
wherein the diameter extension device further comprises at least two cap-engagement means configured to engage with a cap of the medicament delivery device.

20. The diameter extension device according to claim 16,
wherein the diameter extension device further comprises at least two distal protrusions;
wherein each distal protrusion comprises a slot; and
wherein the slot defines the guiding means.

21. The diameter extension device according to claim 16,
wherein the diameter extension device comprises at least two pluralities of holes or recesses each arranged in an axial direction; and
wherein the diameter extension device is configured to engage the pluralities of holes or recesses with the surface feature of the housing body for guiding the axial movement of the diameter extension device.

22. The diameter extension device according to claim 21,
wherein the end portions of the slots define a distal stop position and a proximal stop position for the axial movement of the diameter extension device.

23. The diameter extension device according to claim 22,
wherein the cap engagement means are configured to disengage when the diameter extension device is in the proximal stop position.

24. The diameter extension device according to claim 19,
wherein the diameter extension device further comprises at least two flexible arms protruding radially inwardly from the annular flange and configured to engage with the delivery member cover.

25. The diameter extension device according to claim 24,
wherein the flexible arms are configured to be flexed in radially outward and distal direction, when the diameter extension device is in a distal position and/or the cap is attached to the medicament delivery device;
wherein the flexible arms are further configured to be relaxed in a radial direction, when the diameter extension device is in the proximal stop position; and
wherein the flexible arms are further configured to, when being relaxed at the proximal stop position, to engage with the delivery member cover and to move the delivery member cover from the first to the second position, while the diameter extension device is moved in a distal direction.

26. The diameter extension device according to claim 21,
wherein the most proximal of the holes or recesses define a respective distal stopping position of the diameter extension device; and
wherein the most distal of the holes or recesses define a respective proximal stopping portion of the diameter extension device.

27. The diameter extension device according to claim 26,
wherein at the proximal stopping position of the diameter extension device the proximal end of the diameter extension device is at the same position or at a further proximal position as the proximal end of the delivery member cover in the first position; and
wherein at the distal stopping position of the diameter extension device the proximal end of the diameter extension device is at the same position or at a further distal position as the proximal end of the delivery member cover in the second position.

28. A medicament delivery device suitable for the diameter extension device according to claim 16.

29. A system comprising the medicament delivery device according to claim and the diameter extension device according to claim 16.

30. A diameter extension device for a medicament delivery device, wherein the medicament delivery device comprises:
a housing body with a generally tubular structure with a central axis in a proximal-to-distal direction and configured to hold a medicament delivery system and a drive assembly; and
a delivery member cover:
configured to cover at least a proximal portion of the medicament delivery system,
configured to move, when pressed against tissue at an injection site, coaxially with the central axis within the housing body from a first proximal position to at least a second more distal position, and
further configured to activate by movement from the first position towards the second position the drive assembly to initiate medicament delivery; and
at least two structural features recessing or protruding from an outer surface of the housing body; and
wherein the diameter extension device comprises:
a body with a generally tubular structure being configured to surround at least a portion of the housing body;
at least two grabbing means configured to engage with the at least two structural features in order to hold a distal portion of the diameter extension device fix to the housing body; and
a flexible portion configured to allow for axial movement of a proximal portion of the diameter extension device.
